(19) United States
(12) Patent Application Publication
(10) Pub. No.: US 2008/0132782 A1
(43) Pub. Date: Jun. 5, 2008

(54) Combined MR-ultrasound (US) coil for prostate-, cevix- and rectum cancer imaging diagnostics

(76) Inventors: Bogdan von Rueckmann, Munchen (DE); Ralph Gareus, Forchheim (DE); Wilhelm Durr, Erlangen (DE); Ralph Weissleder, Peabody, MA (US); Arne Hengerer, Erlangen (DE)

Correspondence Address: Siemens Corporation;Intellectual Property Department, 170 Wood Avenue South, Iselin, NJ, 08830, US

(21) Appl. No.: 11/045,619

(22) Filed: Jan. 27, 2005

Related U.S. Application Data
(60) Provisional application No. 60/541,022, filed on Feb. 2, 2004.

(51) Int. Cl. A61B 5/055; A61B 8/00

(52) U.S. Cl. 600/423; 600/443

(57) ABSTRACT

We present, in exemplary embodiments of the present invention, a system combining anatomical imaging technologies (e.g., MR) with molecular imaging technologies (e.g., ultrasound). The system can be used for a variety of applications, including, but not limited to, (1) cancer diagnosis and staging; (2) image guidance; and (3) radiation therapy planning. Image guidance may include guiding a biopsy. For example, a prostatectomy potentially has severe side effects, such as impotence and incontinence. Thus, a histologically-confirmed diagnosis, such as one provided from a biopsy, may prevent unnecessary prostatectomy. Image guidance may also include guiding minimal invasive therapy, such as brachytherapy focused ultrasound. The present invention may be used to plan radiation therapy, for example, by detecting, and thus sparing, healthy tissue from radiation exposure.

### CROSS-REFERENCE TO RELATED APPLICATIONS

This application claims priority to U.S. Provisional Application No. 60/541,022, which was filed on Feb. 2, 2004, and which is fully incorporated herein by reference.

### BACKGROUND OF THE INVENTION

1. Field of the Invention

The present invention relates generally to the field of medical imaging, and, more particularly, to combined magnetic resonance-ultrasound coil for prostate, cervix and rectum imaging.

2. Description of the Related Art

Various diagnostic imaging methods are currently used for the diagnosis of prostate, cervix and rectum cancers. Modern diagnostic imaging techniques include magnetic resonance (“MR”), computer tomography (“CT”), ultrasound (“US”) and nuclear medicine (e.g., Positron Emission Tomography (“PET”), Single Photon Emission Computed Tomography (“SPECT”)). A more accurate diagnosis can be provided by combining the different imaging techniques. In particular, anatomical information (e.g., bones and organs) from an anatomical imaging technique may be enriched with molecular information (e.g., malignant vs. benign tissue) from a molecular imaging technique.

The different imaging techniques are typically combined during post-processing. Post-processing is generally time-consuming. Further, accurate combinations of anatomical and molecular modalities are generally only possible with rigid structures, such as the brain. However, even the brain has minor movement, which can potentially render the combinations inaccurate.

### SUMMARY OF THE INVENTION

In one aspect of the present invention, an apparatus for providing anatomical and molecular diagnostic imaging is provided. The apparatus includes an anatomical imaging unit for inserting into a body cavity, wherein the anatomical imaging unit acquires anatomical images of the body cavity; and a molecular imaging unit operatively connected to the anatomical imaging unit, wherein the molecular imaging unit acquires molecular information of the body cavity.

In another aspect of the present invention, an apparatus for providing anatomical and molecular diagnostic imaging is provided. The apparatus includes a sheath encompassing a magnetic resonance coil; and an ultrasound probe operatively connected to one end of the sheath.

In yet another aspect of the present invention, a method is provided. The method includes inserting a combined anatomical-molecular device into a body cavity; and receiving a combined image from the combined anatomical-molecular device, wherein the combined image displays anatomical imaging-based information and molecular imaging-based information

### BRIEF DESCRIPTION OF THE DRAWINGS

The invention may be understood by reference to the following description taken in conjunction with the accompanying drawings, in which like reference numerals identify like elements, and in which:

FIG. 1 depicts an combined MR-US device, in accordance with one exemplary embodiment of the present invention.

### DETAILED DESCRIPTION OF PREFERRED EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims. It is to be understood that the systems and methods described herein may be implemented in various forms of hardware, software, firmware, special purpose processors, or a combination thereof.

We present, in exemplary embodiments of the present invention, a system combining anatomical imaging technologies (e.g., MR) with molecular imaging technologies (e.g., ultrasound). The system can be used for a variety of applications, including, but not limited to, (1) cancer diagnosis and staging; (2) image guidance; and (3) radiation therapy planning. Image guidance may include guiding a biopsy. For example, a prostatectomy potentially has severe side effects, such as impotence and incontinence. Thus, a histologically-confirmed diagnosis, such as one provided from a biopsy, may prevent unnecessary prostatectomy. Image guidance may also include guiding minimal invasive therapy, such as brachytherapy focused ultrasound. The present invention may be used to plan radiation therapy, for example, by detecting, and thus sparing, healthy tissue from radiation exposure.

We propose a combined magnetic resonance-ultrasound (“MR-US”) imaging device which is inserted into an accessible body cavity, such as the rectum or the vagina. An ultrasound (“US”) probe is integrated into a magnetic resonance (“MR”) coil for enriching magnetic resonance-based images with ultrasound-based information.

The combined MR-US device may be inserted into a body cavity and a combined image displaying anatomical imaging-based information and optical imaging-based information is received. The combined image may be used for any of a variety of practical applications, as contemplated by those skilled in the art, such as diagnostics (e.g., cancer diagnostics), application guiding and therapy planning.

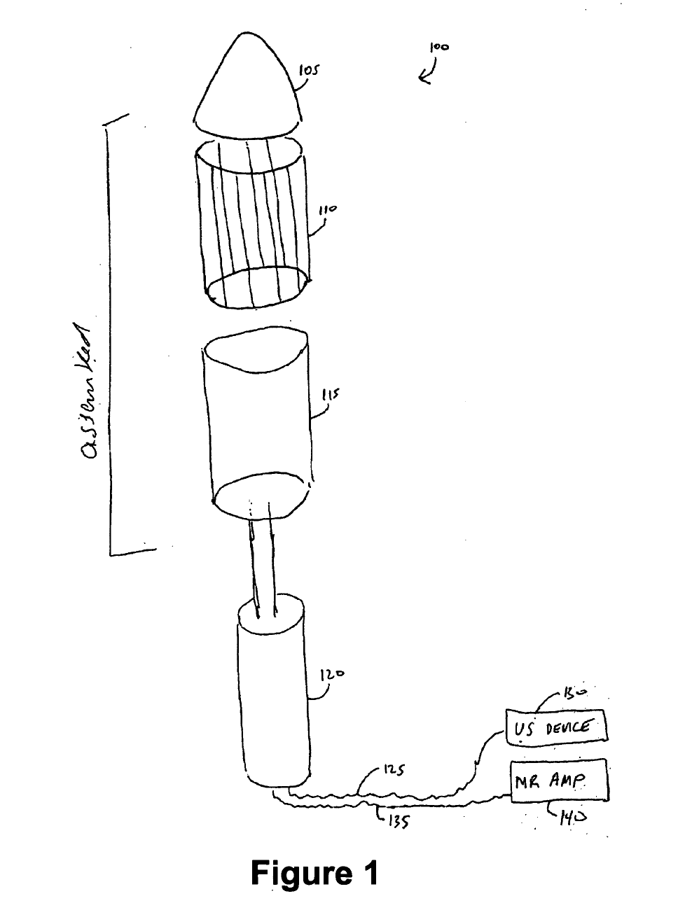

Referring now to FIG. 1, an exploded view of a MR-US device 100 with combined MR imaging and US imaging functionality is shown, in accordance with one exemplary embodiment of the present invention. The optical-MR device 100 includes a US probe 105, an MR coil 110, a sheath 115, and a handheld component 120. The US probe 105 may be a trans-rectal ultrasound transducer. The US probe 105 may be pushed and/or pulled relative to the MR coil. The US probe 105 may also rotate independently of the MR coil 110. The handheld component 120 allows a user to easily grip and handle the MR-US device 100.

The sheath 115 comprises a rigid housing, preferably transluminescent and filled with a coupling liquid (not shown). The coupling liquid preferably has the same optical index as the surrounding material to prevent bending of illumination light beams (described in greater detail below).

The MR-US device 100 includes a US wire 125 operatively connected to a US device 130. The MR-US device 100 further includes an MR wire 135 operatively connected to an amplifier 140. The US wire 125 and the MR wire 135 may be parallel. It should be appreciated that, although not shown for the sake of simplicity, the amplifier 140 may be a component of a magnetic resonance tomography (“MRT”) unit, as known to those skilled in the art. It should further be appreciated that although only one wire is shown, the US wire and the MR wire may include more than one wire, as contemplated by those skilled in the art. The MR coil 110 is preferably constructed to be translucent (e.g., the spacing between the wires may be made out of a translucent plastic, the coil wires are constructed to be stable without a matrix/support).

In an alternate embodiment, the sheath 115 may be replaced with a light transparent balloon (not shown). The balloon may comprise a foldable investigation head with a liquid pump that fuels rotation. The sheath 115 and the balloon encompass the assembly of MR coil 110. The sheath 115 and the balloon may fix the MR-US device 100 to the body cavity. For example, when the MR-US device 100 is inserted in the body cavity, the balloon may be inflated. The balloon is preferably inflated with a coupling liquid, but, in an alternate embodiment, may be inflated with air. If a transparent balloon is used, the MR coil 110 (e.g., RF coil, bird cage) may be a flexible coil, such as the MRInnervu® commercially distributed by MEDRAD® Incorporated.

Furthermore, the sheath 115 and the balloon may be any of a variety of shapes (e.g. toric) as contemplated by those skilled in the art. The sheath 115 and the balloon are preferably shaped such that the MR-US device 110 can easily penetrate the particular body cavity being examined. Although only one balloon is described here, it should be appreciated that more than one (e.g., two) balloons may be used, as contemplated by those skilled in the art.

The intra-rectal measurements obtained from the MR-US device 100 are preferably combined with an external phased array coils (not shown) for increasing signal-to-noise and increasing the area of the body cavity being examined. The external phased array coils are a typical component of a standard MRT unit. In one embodiment, the external phased array coils may be body arrays, providing ventral and dorsal receiver channels. The body arrays expand the viewable area in the body cavity, which provides information of lymph nodes for, for example, staging.

The MR-US device 100 may also be combined with monocrystalline iron oxide nanoparticles (“MION”), which is an MR contrast agent. The MION may be dually labeled with a fluorescence dye, which is an optical contrast agent. Further, the MR data set (i.e., the image obtained from the MRT unit using the present invention) may be used for segmentation and subsequence modeling of NIRF absorption and auto-florescence for increasing the NIRF image quality.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed is:

1. An apparatus for providing anatomical and molecular diagnostic imaging, comprising:
an anatomical imaging unit for inserting into a body cavity, wherein the anatomical imaging unit acquires anatomical images of the body cavity; and
a molecular imaging unit operatively connected to the anatomical imaging unit, wherein the molecular imaging unit acquires molecular information of the body cavity.

2. The apparatus of claim 1, wherein the anatomical imaging unit comprises a magnetic resonance component.

3. The apparatus of claim 1, wherein the molecular imaging unit comprises an ultrasound component.

4. The apparatus of claim 1, wherein the molecular imaging unit moves relative to the anatomical imaging unit.

5. The apparatus of claim 1, wherein the molecular imaging unit rotates independent of the anatomical imaging unit.

6. The apparatus of claim 1, wherein movement of the molecular imaging unit is reproducible.

7. The apparatus of claim 1, further comprising a handheld component operatively connected to the anatomical imaging unit and the molecular imaging unit.

8. An apparatus for providing anatomical and molecular diagnostic imaging, comprising:
a sheath encompassing a magnetic resonance coil; and
an ultrasound probe operatively connected to one end of the sheath.

9. The apparatus of claim 8, further comprising a handheld component operatively connected to the other end of the sheath.

10. The apparatus of claim 8, wherein the magnetic resonance coil is a flexible coil.

11. The apparatus of claim 8, wherein the sheath comprises one of a rigid housing and at least one balloon.

13. The apparatus of claim 8, wherein the ultrasound probe revolves independently of the MR coil.

14. The apparatus of claim 8, wherein the magnetic resonance coil is operatively connected to a magnetic resonance amplifier.

15. The apparatus of claim 8, wherein the ultrasound probe is operatively connected to an ultrasound device.

16. The apparatus of claim 8, further comprising a phased array of coils for increasing signal-to-noise ratio and increasing the region of investigation.

17. A method, comprising:
inserting a combined anatomical-molecular device into a body cavity; and
receiving a combined image from the combined anatomical-molecular device, wherein the combined image displays anatomical imaging-based information and molecular imaging-based information.

18. The method of claim 17, wherein the step of inserting a combined anatomical-molecular device comprises inserting a combined MR-US device.

19. The method of claim 17, the step of inserting a combined anatomical-molecular device into a body cavity comprises inserting a combined anatomical-molecular device into a rectum.

20. The method of claim 17, further comprising analyzing the combined image for one of diagnostics, application guiding and therapy planning
